(19) United States
(12) Patent Application Publication
(10) Pub. No.: US 2025/0027053 A1
(43) Pub. Date: Jan. 23, 2025

(54) METHOD OF CULTURING STEM CELLS BY USING DOUBLE-LAYER COMPOSITE HYDROGEL MICROCARRIER

(71) Applicant: Chun-Hsuan HO, Taipei City (TW)

(72) Inventor: Chun-Hsuan HO, Taipei City (TW)

(21) Appl. No.: 18/641,528

(22) Filed: Apr. 22, 2024

(30) Foreign Application Priority Data
Jul. 19, 2023 (TW) 112126994
Feb. 22, 2024 (CN) 202410196600.3

(51) Int. Cl. C12N 5/074

(52) U.S. Cl. CPC C12N 5/0696; C12N 2531/00; C12N 2533/30; C12N 2533/78

(57) ABSTRACT

A method of culturing a stem cell by using a double-layer composite hydrogel microcarrier is provided. The double-layer composite hydrogel microcarrier includes: an inner-layer hydrogel structure, formed by ionic crosslinking of an inner-layer polymer through an inner-layer cross-linker, in which the inner-layer polymer includes a first inner-layer polymer and a second inner-layer polymer, the first inner-layer polymer is sodium alginate, the second inner-layer polymer is carboxymethyl cellulose, a weight ratio of the sodium alginate and the carboxymethyl cellulose is 3:2, and a weight percentage of the carboxymethyl cellulose based on 100% by weight percentage of the inner-layer hydrogel structure is greater than 1%; and an outer-layer hydrogel structure, formed by covalent crosslinking of an outer-layer monomer through an outer-layer cross-linker, in which the outer-layer hydrogel structure encapsulates the inner-layer hydrogel structure, and the nutrient ingredient is located inside the inner-layer hydrogel structure.

### CROSS-REFERENCE TO RELATED APPLICATION

This application claims priority to Taiwan Application Serial Number 112126994, filed Jul. 19, 2023, and China Application Serial Number 202410196600.3, filed Feb. 22, 2024, which is herein incorporated by reference.

### BACKGROUND

### Field of Invention

The present disclosure is related to a double-layer composite hydrogel microcarrier, manufacture method and use thereof. Particularly, the present disclosure is related to the use of the double-layer composite hydrogel microcarrier for culturing a stem cell.

### Description of Related Art

Gel has the characteristics of releasing the contents loaded inside and delaying release, and are widely used in the field of drug release. However, conventional gels have limitations such as collapse easily and insufficient structural strength, making it difficult to provide long-term and stable release of contents. Therefore, the culture efficiency is limited when the gel is used to culture stem cells.

Therefore, how to provide a gel with enhanced structural strength and prolonged release time to improve culture efficiency of stem cells is a problem to be solved.

### SUMMARY

Some embodiments of the present disclosure provides a method of culturing a stem cell by using a double-layer composite hydrogel microcarrier, including: providing a stem cell, a double-layer composite hydrogel microcarrier and a nutrient ingredient. The double-layer composite hydrogel microcarrier includes: an inner-layer hydrogel structure, formed by ionic crosslinking of an inner-layer polymer through an inner-layer cross-linker, in which the inner-layer polymer includes a first inner-layer polymer and a second inner-layer polymer, the first inner-layer polymer is sodium alginate, the second inner-layer polymer is carboxymethyl cellulose, a weight ratio of the sodium alginate and the carboxymethyl cellulose is 3:2, and a weight percentage of the carboxymethyl cellulose is greater than 1% based on 100% by weight percentage of the inner-layer hydrogel structure; and an outer-layer hydrogel structure, formed by covalent crosslinking of an outer-layer monomer through an outer-layer cross-linker, in which the outer-layer hydrogel structure encapsulates the inner-layer hydrogel structure, in which the nutrient ingredient is located inside the inner-layer hydrogel structure; mixing the stem cell, the double-layer composite hydrogel microcarrier and the nutrient ingredient to obtain a gelation culture medium; and adding a culture fluid to the gelation culture medium.

In some embodiments, the stem cell includes an embryonic stem cell, a hematopoietic stem cell, a mammary stem cell, a mesenchymal stem cell, an endothelial stem cell, a neural stem cell, an olfactory stem cell, an adipose stem cell or a combination thereof.

In some embodiments, the inner-layer hydrogel structure is represented as a plurality of inner sheet structures, and the plurality of inner sheet structures are connected to each other and separated from each other by a plurality of inner-layer holes; and the outer-layer hydrogel structure is represented as a plurality of outer sheet structures, and the plurality of outer sheet structures are connected to each other and separated from each other by a plurality of outer-layer holes, in which a hole diameter of each of the plurality of outer-layer holes is less than a hole diameter of each of the plurality of inner-layer holes.

In some embodiments, the inner-layer hydrogel structure is represented as an interpenetrating network of the first inner-layer polymer and the second inner-layer polymer.

In some embodiments, the inner-layer polymer and the inner-layer cross-linker have opposite electrical properties.

In some embodiments, the outer-layer monomer includes N,N-dimethylacrylamide, acrylamide or a combination thereof.

In some embodiments, the outer-layer cross-linker includes N,N′-methylenebisacrylamide.

In some embodiments, a weight percentage of the sodium alginate is from 0.1% to 5% based on 100% by weight percentage of the inner-layer hydrogel structure.

In some embodiments, a weight percentage of the carboxymethyl cellulose is from 1% to 5% based on 100% by weight percentage of the inner-layer hydrogel structure.

In some embodiments, the nutrient ingredient includes growth factor, tretinoin, ampicillin, bovine serum albumin or a combination thereof.

Some embodiments of the present disclosure provides a method of culturing a stem cell by using a double-layer composite hydrogel microcarrier, including: providing a stem cell, a double-layer composite hydrogel microcarrier and a nutrient ingredient, in which the nutrient ingredient includes macromolecular protein with a molecular weight of at least 500 g/mol, the double-layer composite hydrogel microcarrier includes: an inner-layer hydrogel structure, formed by ionic crosslinking of an inner-layer polymer through an inner-layer cross-linker, in which the inner-layer polymer includes a first inner-layer polymer and a second inner-layer polymer, the first inner-layer polymer is sodium alginate, the second inner-layer polymer is carboxymethyl cellulose, a weight ratio of the sodium alginate and the carboxymethyl cellulose is 3:2; and an outer-layer hydrogel structure, formed by covalent crosslinking of an outer-layer monomer through an outer-layer cross-linker, in which the outer-layer hydrogel structure encapsulates the inner-layer hydrogel structure, in which the nutrient ingredient is located inside the inner-layer hydrogel structure; mixing the stem cell, the double-layer composite hydrogel microcarrier and the nutrient ingredient to obtain a gelation culture medium; and adding a culture fluid to the gelation culture medium.

In some embodiments, the stem cell includes an embryonic stem cell, a hematopoietic stem cell, a mammary stem cell, a mesenchymal stem cell, an endothelial stem cell, a neural stem cell, an olfactory stem cell, an adipose stem cell or a combination thereof.

In some embodiments, the inner-layer hydrogel structure is represented as a plurality of inner sheet structures, and the plurality of inner sheet structures are connected to each other and separated from each other by a plurality of inner-layer holes; and the outer-layer hydrogel structure is represented as a plurality of outer sheet structures, and the plurality of outer sheet structures are connected to each other and separated from each other by a plurality of outer-layer holes, in which a hole diameter of each of the plurality of outer-layer holes is less than a hole diameter of each of the plurality of inner-layer holes.

In some embodiments, the inner-layer hydrogel structure is represented as an interpenetrating network of the first inner-layer polymer and the second inner-layer polymer.

In some embodiments, the inner-layer polymer and the inner-layer cross-linker have opposite electrical properties.

In some embodiments, the outer-layer monomer includes N,N-dimethylacrylamide, acrylamide or a combination thereof.

In some embodiments, the outer-layer cross-linker includes N,N′-methylenebisacrylamide.

In some embodiments, a weight percentage of the sodium alginate is from 0.1% to 5% based on 100% by weight percentage of the inner-layer hydrogel structure.

In some embodiments, a weight percentage of the carboxymethyl cellulose is greater than 1% based on 100% by weight percentage of the inner-layer hydrogel structure.

In some embodiments, the weight percentage of the carboxymethyl cellulose is from 1% to 5% based on 100% by weight percentage of the inner-layer hydrogel structure.

### BRIEF DESCRIPTION OF THE DRAWINGS

In order to allow the above-mentioned and other purposes, features, advantages and embodiments of the present disclosure to be more clearly understood, accompanying drawing is described as follows:

FIG. 1 represents a gel state change diagram corresponding to changes in concentration and temperature when ABA type three-block copolymer of poly(ε-caprolactone-co-glycolide) and poly(ethylene glycol (Tri-PCG) is maintained in phosphate buffered saline in some embodiments of the present disclosure.

FIG. 2 represents electron microscopy images of the double-layer composite hydrogel microcarrier in some embodiments of the present disclosure, in which a to c in the upper row are SA-CMC@PDMA-1 group, and d to f in the second row are SA-CMC@PDMA-3 group, g to i in the bottom row are SA-CMC@PAA-1 group.

FIG. 3A represents water absorption swelling characteristics of different inner-layer hydrogel structures.

FIG. 3B represents water absorption swelling characteristics of double-layer composite hydrogel microcarriers with different hydrogel structures.

FIG. 4A, FIG. 4B, FIG. 5A, FIG. 5B, FIG. 6A, and FIG. 6B represent cumulative release ratios of contents of double-layer composite hydrogel microcarriers with different hydrogel structures in different pH, respectively.

FIG. 7 represents a relationship diagram between viscosity and temperature of gel structures with different weight percentages of sodium alginate and carboxymethyl cellulose.

FIG. 8A represents mRNA expression ratios of lipoprotein lipase (LPL) and glyceraldehyde-3-phosphate dehydrogenase (GAPDH) in adipose stem cells when culturing the adipose stem cells using gel structures with different weight ratios of sodium alginate and carboxymethyl cellulose.

FIG. 8B represents cell growth status diagrams under microscope after culturing adipose stem cells using gel structures with different weight ratios of sodium alginate and carboxymethyl cellulose and staining the adipose stem cells purple-red.

### DETAILED DESCRIPTION

It is to be understood that different implementations or embodiments provided in the following may implement different features of the subject matter of the present disclosure. The embodiments of specific components and arrangements are used to simplify the disclosure and not to limit the disclosure. Of course, these are only examples and are not intended to be limiting. For example, the description below that the first feature is formed on the second feature includes the two being in direct contact, or there are other additional features between the two that are not in direct contact. Furthermore, the present disclosure may repeat reference numerals and/or symbols in the various embodiments. Such repetition is for simplicity and clarity and does not represent a relationship between the various embodiments and/or configurations discussed.

As used herein, unless the context specifically dictates otherwise, “a” and “the” may mean a single or a plurality. It will be further understood that “comprise”, “include”, “have”, and similar terms as used herein indicate described features, regions, integers, steps, operations, elements and/or components, but not exclude other features, regions, integers, steps, operations, elements, components and/or groups.

As used herein, the term “about” means that the value of a given quantity varies within 5% of the value (for example, +1%, +2%, +3%, +4%, 5% of the value). These values are examples only and are not intended to be limiting. It should be understood that the term “about” may mean a percentage of the value of a given quantity as interpreted by one skilled in the relevant art in light of the teachings herein.

As used herein, unless otherwise defined, “%” is referred as weight percentage (wt %).

Although a series of operations or steps are described below to illustrate the method disclosed herein, the order of the operations or steps is not to be construed as limiting. For example, certain operations or steps may be performed in a different order and/or concurrently with other steps. In addition, not all illustrated operations, steps, and/or features are required to implement embodiments of the present disclosure. Moreover, each of the operations or steps described herein can include a plurality of sub-steps or actions.

Some embodiments of the present disclosure provide a double-layer composite hydrogel microcarrier, including an inner-layer hydrogel structure and an outer-layer hydrogel structure. The inner-layer hydrogel structure is formed by ionic crosslinking of an inner-layer polymer through an inner-layer cross-linker. The outer-layer hydrogel structure is formed by covalent crosslinking of an outer-layer monomer through an outer-layer cross-linker, in which the outer-layer hydrogel structure encapsulates the inner-layer hydrogel structure.

The structure that the inner-layer hydrogel structure of the double-layer composite hydrogel microcarrier is formed by ionic crosslinking exists the limitations that the structure is easily affected by pH of the environment, or collapses due to weakening of ionic force caused by water absorption and swelling. The structure that the outer-layer hydrogel structure of the double-layer composite hydrogel microcarrier is formed by covalent crosslinking can provide physical support for the inner-layer hydrogel structure, avoid the structural instability of the inner-layer hydrogel structure, and improve the structural strength of the double-layer composite hydrogel microcarrier. Furthermore, the outer-layer hydrogel structure can also serve as a barrier layer to further delay the release time of the contents carried inside (such as nutritional ingredients) and reduce the phenomenon of burst release, so that the contents carried inside can be released stably and kept long-term release.

In some embodiments, the outer-layer hydrogel structure represents porous film-like and covers the inner-layer hydrogel structure to encapsulate the inner-layer hydrogel structure, in which a thickness of the outer-layer hydrogel structure is less than that of the inner-layer hydrogel structure, there are van der waals forces attracting each other between the outer-layer hydrogel structure and the inner-layer hydrogel structure, but there is no covalent bonding between the two hydrogel structures.

In some embodiments, the inner-layer hydrogel structure represents a plurality of inner sheet structures, and the plurality of inner sheet structures are connected to each other and separated from each other by a plurality of inner-layer holes. The outer-layer hydrogel structure is represented as a plurality of outer sheet structures, and the plurality of outer sheet structures are connected to each other and separated from each other by a plurality of outer-layer holes, in which a hole diameter of each of the plurality of outer-layer holes is less than a hole diameter of each of the plurality of inner-layer holes. Through the difference in hole diameter between the inner-layer hydrogel structure and the outer-layer hydrogel structure, the outer-layer hydrogel structure can have better structural strength, and through the design of the small hole diameter of the outer-layer hydrogel structure, it can provide the contents of the double-layer composite hydrogel microcarrier better sustained release effect.

In some embodiments, in the cross-sectional view, each inner sheet structure (or each outer sheet structure) is arc-shaped, and the inner sheet structures are concavely opposed to each other, thus defining the inner-layer holes (or outer-layer holes). In some embodiments, a hole diameter of the inner-layer hole represents a longest vertical distance between two inner sheet structures located on opposite sides of the inner-layer holes. A hole diameter of the outer-layer hole represents a longest vertical distance between two outer sheet structures located on opposite sides of the outer-layer holes. In some embodiments, a hole diameter of the inner-layer hole is from 250 μm to 1000 μm, such as 250 μm, 500 μm, 750 μm, 1000 μm or a value in the aforementioned interval. In some embodiments, a hole diameter of the outer-layer hole is from 50 μm to 500 μm, such as 50 μm, 100 μm, 150 μm, 200 μm, 250 μm, 300 μm, 350 μm, 400 μm, 450 μm, 500 μm or a value in the aforementioned interval. If the hole diameter is too large, the structure will be unstable and the content release rate will be too fast, making it impossible to achieve sustained release; if the hole diameter is too small, the content carried inside will be excessively blocked, making it difficult to achieve stable and long-lasting release of the content.

In some embodiments, a thickness of the outer-layer hydrogel structure is from 25 μm to 1000 μm, such as 25 μm, 50 μm, 100 μm, 200 μm, 300 μm, 400 μm, 500 μm, 600 μm, 700 μm, 800 μm, 900 μm, 1000 μm or a value in the aforementioned interval. If the thickness is too thick, the release rate of the content loaded inside will be too slow; if the thickness is too thin, the release rate of the content loaded inside will be too fast and the structural strength will be insufficient. It can be understood that as the thickness increases, the release of the content (such as lipid soluble small molecule (molecular weight is lower than 500 g/mol, solubility in non-polar solvent is higher than 0.1 μg/1 g) or macromolecular protein (molecular weight is at least higher than 500 g/mol) can be delayed.

In some embodiments, the inner-layer polymer includes a first inner-layer polymer and a second inner-layer polymer, and the inner-layer hydrogel structure represents interpenetrating networks of the first inner-layer polymer and the second inner-layer polymer (that is, polymer chains of the first inner-layer polymer and polymer chains of the second inner-layer polymer are intertwined with each other). Through the design of the interpenetrating networks, it can provide better support and stabilize the structural strength of the inner-layer hydrogel structure.

In some embodiments, the inner-layer polymer and the inner-layer cross-linker have opposite electrical properties. For example, the inner-layer polymer has a negative charge, and the inner-layer cross-linker has a positive charge. The inner-layer cross-linker and the inner-layer polymer are cross-linked by ionic bonding formed by the positive and negative charges to form the inner-layer hydrogel structure; or the inner-layer polymer has a positive charge, and the inner-layer cross-linker has a negative charge. When the inner-layer polymer has different degrees of dissociation at different pH, the inner-layer hydrogel structure formed by cross-linking of ionic bonding is pH sensitive and will change the strength of the ionic bonding due to changes in pH, thereby changing the inner-layer hydrogel structure.

In some embodiments, when the first inner-layer polymer is sodium alginate (SA), the second inner-layer polymer is carboxymethyl cellulose (CMC). The use of sodium alginate and carboxymethyl cellulose has the advantages of being easy to preserve and be prepared, safe and non-toxic, gel formation at room temperature, and being absorbed easily by the human body. In addition, compared with sodium alginate only, the combination of sodium alginate and carboxymethyl cellulose can improve the viscosity and structural strength of the inner-layer hydrogel structure. Furthermore, by adjusting the ratio of sodium alginate and carboxymethyl cellulose, the hole diameters of the inner-layer holes can be adjusted and better flexibility is provided to select the inner-layer hydrogel structure. In addition, since sodium alginate and carboxymethyl cellulose have carboxyl groups, sodium alginate and carboxymethyl cellulose are not easily dissociated in an acidic environment when sodium alginate and carboxymethyl cellulose are served as the materials of the inner-layer hydrogel structure. Therefore, the inner-layer hydrogel structure is denser in the acidic environment. Relatively, sodium alginate and carboxymethyl cellulose are easily dissociated in an alkaline environment. Therefore, the inner-layer hydrogel structure is more loose in the alkaline environment.

In some embodiments, a weight percentage of sodium alginate is from 0.1% to 5% based on 100% by weight percentage of the inner-layer hydrogel structure, such as 0.1%, 0.25%, 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, or a value in the aforementioned interval. A weight percentage of carboxymethyl cellulose is from 0.1% to 5%, such as 0.1%, 0.25%, 0.5%, 0.75%, 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, or a value in the aforementioned interval. If the weight percentages of both are too high, the inner-layer hydrogel structure is too denser, and the release efficiency of the inner-layer hydrogel structure is limited. If the weight percentages of both are too low, the hydrogel structure is loose and unstable, or the hydrogel cannot be formed. In addition, along with increase of the weight percentage of sodium alginate and decrease of the weight percentage of carboxymethyl cellulose, the hole diameters of the inner-layer holes will be increased, the swelling ratio of the inner-layer hydrogel structure during water absorption and the release efficiency of contents are increased. Relatively, along with decrease of the weight percentage of sodium alginate and increase of the weight percentage of carboxymethyl cellulose, the hole diameters of the inner-layer holes will be reduced, the swelling ratio of the inner-layer hydrogel structure and the release efficiency are reduced (such as, reduce of the release of the macromolecular protein).

In some embodiments, when the first inner-layer polymer is copolymer of poly(ε-caprolactone-co-glycolic acid) and poly(ethylene glycol), the second inner-layer polymer is a derivative of copolymer of poly(ε-caprolactone-co-glycolic acid) and poly(ethylene glycol) that has an acyl group. In one embodiment, copolymer of poly(ε-caprolactone-co-glycolic acid) and poly(ethylene glycol) includes ABA type three-block copolymer of poly(ε-caprolactone-co-glycolic acid) and poly(ethylene glycol) (Tri-PCG). Therefore, the derivative of copolymer of poly(ε-caprolactone-co-glycolic acid) and poly(ethylene glycol) that has an acyl group is Tri-PCG with an acrylate group (Tri-PCG-acryl). It is worth emphasizing that the inner-layer hydrogel structure of Tri-PCG and Tri-PCG-acryl can change the hydrogel properties according to changes in temperature within the suitable temperature range of the human body (for example, 25° C. to 37° C., from solution form to gel form), thereby changing viscosity and release ability of the inner-layer hydrogel structure, and the structure is more stable and difficult to collapse. In some embodiments, the double-layer composite hydrogel microcarrier further includes inner auxiliary molecule existed in the inner-layer hydrogel structure (such as dipentaerythritolhexakis (3-mercaptopropionate) (DPMP)). Along with the increase of the temperature, DPMP can form a covalent structure with Tri-PCG-acryl, thus, gel properties can be enhanced, and temperature sensitivity of the inner-layer hydrogel structure is increased.

In some embodiments, a total weight percentage of Tri-PCG and Tri-PCG-acryl is 15% to 30% based on 100% by weight percentage of the inner-layer hydrogel structure, such as 15%, 16%, 17%, 18%, 19%, 20%, 21%, 22%, 23%, 24%, 25%, 26%, 27%, 28%, 29%, 30%, or a value in the aforementioned interval. If the weight percentage is too high, the inner-layer hydrogel structure is too denser, and the release efficiency of the inner-layer hydrogel structure is limited. If the weight percentage is too low, the gel structure is loose and unstable, or the hydrogel cannot be formed.

In some embodiments, when the inner-layer polymer has negative charges, the inner-layer cross-linker includes calcium ions (Ca²⁺), which can cross-link with the inner-layer polymer. Compared with other inner-layer cross-linker, calcium ions have the advantages of being easy to obtain, low cost, and less harmful to the environment.

In some embodiments, the outer-layer monomer includes N,N-dimethylacrylamide (DMAA), acrylamide (AA) or a combination thereof. Compared with AA, when DMAA is selected, hole diameters of outer-layer holes of the formed outer-layer hydrogel structure are larger, the release ratio of the contents carried inside is higher (such as increase of the release of macromolecular protein), thereby achieving higher swelling ratio of the double-layer composite hydrogel microcarrier when water absorption.

In some embodiments, the outer-layer cross-linker includes N,N′-Methylene-Bis-Acrylamide (BIS), which can form covalent crosslinking with DMAA or AA, thereby crosslinking DMAA to form poly(N,N-dimethylacrylamide) (PDMA), or crosslinking AA to form poly(acrylamide) (PAA).

Some embodiments of the present disclosure provide a method of manufacturing a double-layer composite hydrogel microcarrier, including: providing an inner-layer polymer and an inner-layer cross-linker; mixing an inner-layer polymer and an inner-layer cross-linker to obtain an inner-layer hydrogel structure by ionic crosslinking the inner-layer polymer; providing an outer-layer monomer and outer-layer cross-linker; mixing an inner-layer hydrogel structure and an outer-layer monomer and outer-layer cross-linker to obtain an outer-layer hydrogel structure by covalently crosslinking the outer-layer monomer and the outer-layer cross-linker and encapsulating the inner-layer hydrogel structure. By mixing the inner-layer hydrogel structure, the outer-layer monomer and the outer-layer cross-linker, the outer-layer hydrogel structure encapsulating the inner-layer hydrogel structure can be formed, the outer-layer hydrogel structure can provide physical support for the inner-layer hydrogel structure, improve structure strength of the double-layer composite hydrogel microcarrier, delay the release time of the contents carried inside and achieve stable and long-term release.

In some embodiments, the step of providing the inner-layer polymer and inner-layer cross-linker includes providing a first inner-layer polymer, a second inner-layer polymer and an inner-layer cross-linker.

In some embodiments, the first inner-layer polymer is sodium alginate (SA), and the second inner-layer polymer is carboxymethyl cellulose (CMC), and the use of sodium alginate and carboxymethyl cellulose has the advantages of being easy to preserve and prepare, safe and non-toxic, gel formation at room temperature, and being absorbed easily by the human body.

In some embodiments, when the first inner-layer polymer is copolymer of poly(ε-caprolactone-co-glycolic acid) and poly(ethylene glycol), the second inner-layer polymer is a derivative of copolymer of poly(ε-caprolactone-co-glycolic acid) and poly(ethylene glycol) that has an acyl group. In one embodiment, copolymer of poly(ε-caprolactone-co-glycolic acid) and poly(ethylene glycol) includes ABA type three-block copolymer of poly(ε-caprolactone-co-glycolic acid) and poly(ethylene glycol) (Tri-PCG). The inner-layer hydrogel structure manufactured by Tri-PCG and Tri-PCG-acryl is much denser and the hydrogel properties can be changed according to changes in temperature within the suitable temperature range of the human body (for example, 25° C. to 37° C., from solution form to gel form), thereby changing viscosity and release ability of the inner-layer hydrogel structure, and the structure is more stable and difficult to collapse.

In some embodiments, the inner-layer polymer and inner-layer cross-linker have opposite electrical properties. In some embodiments, when the inner-layer polymer has negative charges, the inner-layer cross-linker includes calcium ions.

In some embodiments, the step of mixing the inner-layer polymer and the inner-layer cross-linker includes the first inner-layer polymer, the second inner-layer polymer, the inner-layer cross-linker, a free radical generator and water. By adding the free radical generator at the step, the free radical generator stored in the inner-layer hydrogel structure can diffuse to the outer layer and react with the outer-layer monomer, the outer-layer cross-linker and the catalyst when the outer-layer monomer, the outer-layer cross-linker and the catalyst are subsequently added, thereby inducing the reaction of forming an outer-layer hydrogel structure in the outer layer. Therefore, the addition of the free radical generator at the step can prevent the pre-mature reaction when the free radical generator and the outer-layer monomer, the outer-layer cross-linker and the catalyst are co-added, or prevent the situation that the outer-layer hydrogel structure does not encapsulate the inner-layer hydrogel structure well.

In some embodiments, a weight ratio of the first inner-layer polymer and the second inner-layer polymer is from 1:5 to 5:1, such as 1:5, 1:4, 1:3, 1:2, 1:1, 2:1, 3:1, 4:1, 5:1, or a value in the aforementioned interval. Compared with the acid environment, when the first inner-layer polymer has more free negatively charged groups (such as carboxyl groups) in the alkaline environment, the swelling ratio of the inner-layer hydrogel structure is higher along with the increase of the weight ratio.

In some embodiments, the step of mixing the inner-layer polymer and the inner-layer cross-linker includes mixing the first inner-layer polymer, the second inner-layer polymer, the inner-layer cross-linker, the free radical generator and water.

In some embodiments, a weight percentage of sodium alginate is from 0.1% to 5%, and a weight percentage of carboxymethyl cellulose is from 0.1% to 5% based on 100% by total weight percentage of the first inner-layer polymer, the second inner-layer polymer, the inner-layer cross-linker, the free radical generator and water. If the weight percentage is too high, the inner-layer hydrogel structure is too denser, the release efficiency of the inner-layer hydrogel structure is limited; if the weight percentage is too low, the hydrogel structure is unstable, or the hydrogel cannot be formed. The weight percentage of carboxymethyl cellulose higher than 1% (such as 1%, 1.25%, 1.5%, 1.75%, 2%, 2.25%, 2.5%, 2.75%, 3%, 3.25%, 3.5%, 3.75%, 4%, 4.25%, 4.5%, 4.75%, 5%, or a value in the aforementioned interval) provides the double-layer composite hydrogel microcarrier with enough structure strength for use in culturing the stem cells several days without collapse. In some embodiments, a weight ratio of sodium alginate and carboxymethyl cellulose of 3:2 controls the hole diameters of the inner-layer hydrogel structure to allow the nutrient ingredient to be stably released at suitable rates, thereby ensuring that the stem cell intakes the nutrient ingredient stably, increasing the growth rate of the stem cells.

In some embodiments, a total weight percentage of copolymer of poly(ε-caprolactone-co-glycolic acid) and poly(ethylene glycol) and the derivative of copolymer of poly(ε-caprolactone-co-glycolic acid) and poly(ethylene glycol) that has an acyl group is from 15% to 30% based on 100% by weight percentage of the first inner-layer polymer, the second inner-layer polymer, the inner-layer cross-linker, the free radical generator and water. If the weight percentage is too high, the inner-layer hydrogel structure is too denser, and the release efficiency of the inner-layer hydrogel structure is limited. If the weight percentage is too low, the gel structure is loose and unstable, or the hydrogel cannot be formed.

In some embodiments, the step of mixing the first inner-layer polymer, the second inner-layer polymer, the inner-layer cross-linker, the free radical generator and water further includes adding dipentaerythritolhexakis (3-mercaptopropionate), bovine serum albumin or a combination thereof. In some embodiments, the step of mixing the first inner-layer polymer, the second inner-layer polymer, the inner-layer cross-linker, the free radical generator and water includes mixing a first solution, a second solution, calcium ions and ammonium sulfate, in which the first solution includes copolymer of poly(ε-caprolactone-co-glycolic acid) and poly(ethylene glycol), or copolymer of poly(ε-caprolactone-co-glycolic acid) and poly(ethylene glycol) and dipentaerythritolhexakis (3-mercaptopropionate) (DPMP), and the second solution includes the derivative of copolymer of poly(ε-caprolactone-co-glycolic acid) and poly(ethylene glycol) that has an acyl group and bovine serum albumin.

It can be understood that as the temperature increases, DPMP tends to form covalent crosslinking with the acyl group, thus increasing the density of the inner-layer hydrogel structure. Therefore, by adding DPMP, the sustained release capability can be enhanced when the temperature rises, which is beneficial for use in the human body. In some embodiments, a weight percentage of DPMP is from 5% to 15% based on 100% by weight percentage of the first solution, such as 5%, 6%, 7%, 8%, 9%, 10%, 11%, 12%, 13%, 14%, 15% or a value in the aforementioned interval. Furthermore, by adding bovine serum albumin, bovine serum albumin can be encapsulated in the double-layer composite hydrogel microcarrier. When the double-layer composite hydrogel microcarrier is used in cell culture, bovine serum albumin can be released continuously, providing a long-lasting source of cell nutrients.

In some embodiments, the step of mixing the inner-layer hydrogel structure, the outer-layer monomer and the outer-layer cross-linker includes mixing an inner-layer hydrogel structure, N,N-dimethylacrylamide (DMAA), N,N′-methylenebisacrylamide (BIS) and the catalyst, or mixing the inner-layer hydrogel structure, acrylamide (AA), N,N′-methylenebisacrylamide and the catalyst. By forming a covalent outer-layer hydrogel structure with BIS and DMAA or AA, physical support can be provide to the inner-layer hydrogel structure, and the denser holes are formed compared with the inner-layer hydrogel structure to prolong the release of the contents carried inside.

In some embodiments, based on 100% by total weight percentage of the inner-layer hydrogel structure, the outer-layer monomer and the outer-layer cross-linker, a weight percentage of the inner-layer hydrogel structure is from 50% to 80% (such as 50%, 60%, 70%, 80% or a value in the aforementioned interval), a weight percentage of the outer-layer monomer is from 15% to 40% (such as 15%, 20%, 25%, 30%, 35%, 40% or a value in the aforementioned interval), and a weight percentage of the outer-layer cross-linker is from 5% to 10% (such as 5%, 6%, 7%, 8%, 9%, 10% or a value in the aforementioned interval). If the weight percentage of the inner-layer hydrogel structure or that of the outer-layer hydrogel structure is too low, the double-layer composite hydrogel microcarrier can be hardly formed. If the weight percentage of the inner-layer hydrogel structure or that of the outer-layer hydrogel structure is too high, the release ratio of the contents carried in the double-layer composite hydrogel microcarrier is too low.

In some embodiments, a weight ratio of the inner-layer hydrogel structure and the outer-layer monomer is from 5:4 to 16:3 (such as 5:4, 5:3, 2:1, 8:3, 10:3, 4:1, 16:3, 16:3 or a value in the aforementioned interval). If the weight ratio is too high, the structure of the double-layer composite hydrogel microcarrier is unstable. If the weight ratio is too low, the outer layer of the double-layer composite hydrogel microcarrier is too thick, and the release ratio of the contents carried inside is too low.

In some embodiments, at the step of mixing the inner-layer hydrogel structure, the outer-layer monomer and the outer-layer cross-linker, the mixing time is from 0.5 min to 5 min, such as 0.5 min, 1 min, 2 min, 3 min, 4 min, 5 min, or a value in the aforementioned interval. By controlling the mixing time, a thickness of the outer-layer hydrogel structure can be controlled, thereby controlling the structure strength and the release ratio of the double-layer composite hydrogel microcarrier.

A method of culturing a stem cell by using an above-mentioned double-layer composite hydrogel microcarrier is provided in some embodiments of the present disclosure, including: providing a stem cell, a double-layer composite hydrogel microcarrier and a nutrient ingredient, in which the double-layer composite hydrogel microcarrier includes: an inner-layer hydrogel structure and an outer-layer hydrogel structure. The inner-layer hydrogel structure is formed by ionic crosslinking of an inner-layer polymer through an inner-layer cross-linker, in which the inner-layer polymer includes a first inner-layer polymer and a second inner-layer polymer, the first inner-layer polymer is sodium alginate, the second inner-layer polymer is carboxymethyl cellulose, a weight ratio of the sodium alginate and the carboxymethyl cellulose is 3:2, and a weight percentage of the carboxymethyl cellulose is greater than 1% based on 100% by weight percentage of the inner-layer hydrogel structure. An outer-layer hydrogel structure is formed by covalent crosslinking of an outer-layer monomer through an outer-layer cross-linker, in which the outer-layer hydrogel structure encapsulates the inner-layer hydrogel structure, and the nutrient ingredient is located inside the inner-layer hydrogel structure. The method also includes mixing the stem cell, the double-layer composite hydrogel microcarrier and the nutrient ingredient to obtain a gelation culture medium; and adding a culture fluid to the gelation culture medium. By controlling a weight percentage of carboxymethyl cellulose, the better structure stability of the double-layer composite hydrogel microcarrier can be provided, and by defining the weight ratio of sodium alginate and carboxymethyl cellulose and the position of the nutrient ingredient, the release time of the nutrient ingredient carried inside can be prolonged. Therefore, it takes longer time for hydrogel to collapse when culturing the stem cell with the double-layer composite hydrogel microcarrier, increasing the culture capacity of the stem cell per unit area and preventing burst release of the contents, and the contents can be released to the stem cells continuously, thereby prolonging the culture time of the stem cells and the time interval required for adding the nutrient ingredient, and simplifying the culture procedure.

In some embodiments, the stem cell includes an embryonic stem cell, a hematopoietic stem cell, a mammary stem cell, a mesenchymal stem cell, an endothelial stem cell, a neural stem cell, an olfactory stem cell, an adipose stem cell or a combination thereof.

It can be understood that the double-layer composite hydrogel microcarriers with different structural strengths and release ratios can be selected according to the different characteristics of the stem cells. For example, when culturing the adipose stem cells with faster growth rate, sodium alginate and carboxymethyl cellulose, providing the larger hole diameters of the inner-layer holes, can be selected. When culturing the neural stem cells with slower growth rate, Tri-PCG and Tri-PCG-acryl, providing the smaller hole diameters of the inner-layer holes, can be selected.

In order to further illustrate the double-layer composite hydrogel microcarrier, the method of manufacturing the double-layer composite hydrogel microcarrier and the method of culturing the stem cell by various embodiments of the present disclosure, the following implementations are preferred. It should be noted that the following examples are provided for illustrative purposes only and do not limit the present disclosure.

### Example 1—Manufacture Method of Double-Layer Composite Hydrogel Microcarrier

### 1. Inner-Layer Hydrogel Structure

The procedures corresponding to different inner-layer polymers were provided below.

### 1.1. Sodium Alginate and Carboxymethyl Cellulose

Sodium alginate (SA) and carboxymethyl cellulose (CMC) were respectively mixed with different weight ratios (SA:CMC=4:1, 2:3, 3:2, 1:4), and then poured into 5% calcium chloride (CaCl₂)) aqueous solution containing 1.5% ammonium sulfate (APS) to form a mixed solution. The mixed solution was stirred for 20 min to allow SA and CMC to crosslink through calcium ions to form several groups of inner-layer hydrogel structures with different concentrations of SA and CMC and obtain inner-layer hydrogels. The concentration of SA and CMC of each group was respectfully 2.0%/0.5%, 1.0%/1.5%, 1.5%/1.0%, and 0.5%/2.0%, the reaction concentration of APS was 1%, and the reaction concentration of calcium ions was 4%.

It could be understood that during the process of forming the mixed solution (not limited to the examples that used sodium alginate and carboxymethyl cellulose as inner-layer polymers), the culture fluid or the nutrient ingredient (such as growth factor) could be added to the mixed solution, and the final concentration of the nutrient ingredient could be ranged from 5 μg/mL to 10 μg/mL for subsequent clinical application according to subsequent culture requirements of the stem cells.

1.2. ABA type three-block copolymer of poly(ε-caprolactone-co-glycolic acid) and poly(ethylene glycol) (Tri-PCG) and Tri-PCG derivative with acrylate groups (or called Tri-PCG-acryl)

### 1.2.1. Manufacture of Tri-PCG

Poly(ethylene glycol) (PEG) (15.0 g, 10 mmol) was placed under reduced pressure and dried at 120° C. for 3 hrs. ε-Caprolactone (33.3 g, 291 mmol), glycolide (5.87 g, 50.6 mmol) and tin 2-ethylhexanoate (Sn(Oct)2) served as catalyst (149 mg, 367 μmol), were added to the flask containing PEG to obtain a reaction solution.

Furthermore, the reaction solution was frozen in liquid nitrogen and dried under reduced pressure to obtain reactants. Subsequently, the flask containing the reactants was immersed in a 160° C. oil bath for 12 hours to perform a ring-opening polymerization reaction. Then, the reactants were dissolved in into 100 mL of chloroform and precipitated with 1000 mL of diethyl ether, and the dissolution step and the precipitation step were repeated three times. Then, the supernatant was removed, and the precipitate was dried under reduced pressure to obtain a white solid, which was Tri-PCG.

### 1.2.2. Manufacture of Tri-PCG-Acryl

Acrylic acid (2.0 mL, 29.2 mmol) was dissolved in 250 ml of dichloromethane (CH₂Cl₂) to obtain an acrylic acid solution; and N,N′-dicyclohexyl carbodiimide (DCC) (6.24 g, 30.2 mmol) was dissolved in 250 ml of methylene chloride to obtain a N,N′-dicyclohexylcarbodiimide solution. Furthermore, in an ice bath, the N,N′-dicyclohexylcarbodiimide solution was added to the acrylic acid solution and stirred for 1 hr, where the volume ratio of the N,N′-dicyclohexylcarbodiimide solution to the acrylic acid solution is 1:1. Then, Tri-PCG (20.3 g, 5.1 mmol) and 4-dimethylaminopyridine (DMAP) (299.5 mg, 2.5 mmol) dissolved in 250 ml of dichloromethane were added to a solution containing acrylic acid and N,N′-dicyclohexylcarbodiimide and stirred for 24 hrs at 25° C. Subsequently, the by-product, dicyclohexylurea, was filtered out. After the methylene chloride evaporated, reprecipitation was performed three times using chloroform as a good solvent (a solvent with better solubility for solutes and an interaction parameter with polymer solutes of less than 0.5), so that the long polymer chains were stretched in the solution, and using the mixture of n-hexane and ethanol (the volume ratio of n-hexane to ethanol is 8/2) as a poor solvent (a solvent with weak solubility for solutes and an interaction parameter with polymer solutes of close to or greater than 0.5). Then, after drying under reduced pressure, Tri-PCG-acryl was obtained as a yellow-white solid.

### 1.2.3. Manufacture of Tri-PCG Aqueous Solution, Tri-PCG-Acryl Aqueous Solution and Tri-PCG/DPMP Aqueous Solution

According to Tri-PCG aqueous solution: first, Tri-PCG (246.5 mg) was dissolved in a culture fluid for adipose stem cells (986 μL, product name: StemPro™ MSC SFM, product number: Gibco™ A1033201) containing 10% bovine serum. After immersing in a water bath of about 90° C. for 5 s, it was continuously stirred and cooled to room temperature, and the steps of the immersion and cooling steps were repeated for three times. Then, it was cooled in water bath condition (from 20° C. to 25° C.), and treated with ultrasonic treatment for 30 min to remove bubbles, and 20% Tri-PCG aqueous solution (may be replaced with subsequent Tri-PCG/DPMP aqueous solution according to hydrogel requirements) was obtained by adjusting pH to 7.4 with sodium hydroxide and the culture fluid for adipose stem cells.

According to Tri-PCG-acryl aqueous solution: Tri-PCG-acryl (165 mg) was dissolved in the culture fluid for adipose stem cells containing 10% bovine serum (660 μL), and stirred overnight at 4° C. Furthermore, it was cooled in water bath condition (from 20° C. to 25° C.), and treated with ultrasonic treatment for 30 min to remove bubbles, and 20% Tri-PCG-acryl aqueous solution was obtained by adjusting pH to 7.4 with sodium hydroxide and the culture fluid for adipose stem cells.

According to Tri-PCG/DPMP aqueous solution: dipentaerythritolhexakis (3-mercaptopropionate) (DPMP) (90.4 mg) was dissolved in 1 mL acetone to obtain DPMP solution. DPMP solution was added to a sample tube containing 750 mg Tri-PCG, and then, 7 mL acetone was added to obtain a mixed solution. The mixed solution was added to 63 mL water, and then the water containing the mixed solution was stirred and treated with ultrasonic treatment for 30 min. Furthermore, acetone was removed and freeze dried to obtain Tri-PCG/DPMP mixture (DPMP is about 10.8% in the Tri-PCG/DPMP mixture).

Furthermore, Tri-PCG/DPMP mixture was dissolved in the culture fluid for adipose stem cells containing 10% bovine serum (354 μL). After immersing in a water bath of about 90° C. for 5 s, it was continuously stirred and cooled to room temperature, and the steps of the immersion and cooling steps were repeated for three times. Furthermore, it was cooled in water bath condition (from 20° C. to 25° C.), and treated with ultrasonic treatment for 30 min to remove bubbles, and 20% Tri-PCG/DPMP aqueous solution was obtained by adjusting pH to 7.4 with sodium hydroxide and the culture fluid for adipose stem cells.

### 1.2.4. Manufacture of Inner-Layer Hydrogel

After mixing Tri-PCG/DPMP aqueous solution and Tri-PCG-acryl aqueous solution with appropriate ratios, 5% calcium chloride (CaCl₂)) aqueous solution containing 1.5% ammonium persulfate was added to form a mixed solution. The mixed solution was stirred for 20 min to allow Tri-PCG and Tri-PCG-acryl to crosslink through calcium ions to form an inner-layer hydrogel structure and obtain an inner-layer hydrogel, in which the reaction concentration of Tri-PCG/DPMP and Tri-PCG-acryl were respectfully from 70% to 79% and from 16% to 25%, the reaction concentration of APS was 1%, the reaction concentration of calcium ions was 4%, and a total weight percentage of Tri-PCG and Tri-PCG-acryl in the inner-layer hydrogel structure was from 16% to 33%, to ensure the inner-layer hydrogel was represented as gel state at operation temperature scope.

It could be understood that as the temperature raised, Tri-PCG and Tri-PCG-acryl or DPMP and acrylate groups of Tri-PCG-acryl formed covalent bonding, thereby changing the gel state. Therefore, the inner-layer gel structure manufactured by Tri-PCG-acryl and Tri-PCG/DPMP, or manufactured by Tri-PCG-acryl and Tri-PCG, had temperature sensitivity.

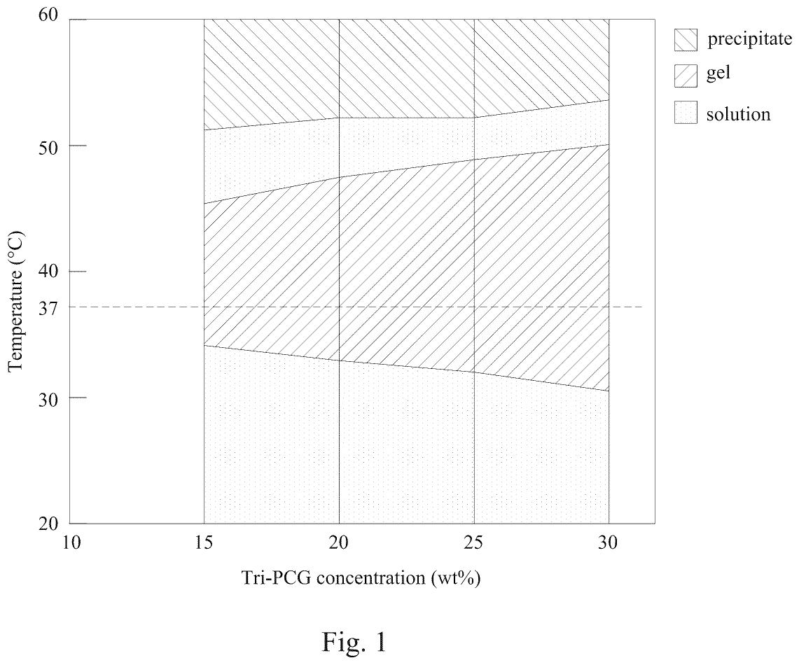

For example, refer to FIG. 1, a gel state change diagram corresponding to changes in concentration and temperature (Tri-PCG example was kept in phosphate buffered saline (PBS), representing that by adjusting the Tri-PCG concentration (for example, corresponding to the total concentrations of Tri-PCG/DPMP and Tri-PCG-acryl in this Example) or temperature, the gel state was changed, and viscosity and release capacity of the hydrogel were adjusted according to the changes of the gel state.

In other examples, Tri-PCG aqueous solution may also be used to replace Tri-PCG/DPMP aqueous solution and mixed with Tri-PCG-acryl. It could be understood that compared with Tri-PCG aqueous solution, Tri-PCG/DPMP aqueous solution contained DPMP that formed covalent bonds with Tri-PCG-acryl when the temperature was raised. Therefore, the inner-layer gel structure manufactured by Tri-PCG/DPMP aqueous solution had better temperature sensitivity.

It could be understood that the inner-layer hydrogel structure manufactured by “1.1-Sodium Alginate and Carboxymethyl Cellulose” and the inner-layer hydrogel structure manufactured by “1.2. ABA type three-block copolymer of poly(¿-caprolactone-co-glycolic acid) and poly(ethylene glycol) (Tri-PCG) and Tri-PCG derivative with acrylate groups (or called Tri-PCG-acryl)” were different from each other in that the inner-layer hydrogel structure using Tri-PCG/DPMP and Tri-PCG-acryl was more stable, relatively stable and difficult to collapse, but the manufacture process was more complicated and the use of the organic solvents was required. As for the inner-layer hydrogel structure using SA and CMC, it had the advantages of simpler manufacture process and was more friendly to the environment, but there was a limitation that the structure was also affected by pH.

Therefore, according to the growth characteristics of the stem cells to be cultured later, appropriate ingredients were selected to prepare the inner-layer hydrogel structure, such as the selection of SA and CMC, which was easily to manufacture the inner-layer hydrogel structure, to culture adipose stem cells with quicker growth rate, or the selection of Tri-PCG/DPMP and Tri-PCG-acryl, which had stronger structure strength, to culture neural stem cells with slower growth rate.

### 2. Outer-Layer Hydrogel Structure and Double-Layer Composite Hydrogel Microcarrier

Furthermore, after mixing N,N-dimethylacrylamide (DMAA) or acrylamide (AA) served the as outer-layer monomer, N,N′-methylenebisacrylamide (BIS) served as the outer-layer cross-linker, and tetra-methylethylenediamine (TEMED) served as the catalyst, they were further mixed with the inner-layer hydrogel manufactured before (the inner-layer hydrogel obtained from “1.1. Sodium Alginate and Carboxymethyl Cellulose” was selected) for different time periods to make DMAA or AA crosslink with BIS to form poly(N,N-dimethylacrylamide) (PDMA) or poly(acrylamide) (PAA) and obtain double-layer composite hydrogel microcarriers with different thickness of the outer-layer hydrogel structure or different outer-layer ingredients, in which the reaction concentration of DMAA or AA was 1 mol/L, the reaction concentration of BIS was 2.2 mol/L, and the reaction concentration of TEMED was 15 μg/mL.

Particularly, during the mixing process, APS in the inner-layer hydrogel diffused to the outside, providing free radicals to initiate the reaction to form the outer-layer hydrogel structure. The outer-layer monomer was covalently bonded to BIS through the free radicals of APS and the catalysis of TEMED to form PDMA or PAA and formed an outer-layer hydrogel structure.

Finally, the unreacted outer-layer monomer was removed by stirring and washing with water.

### Example 2—Method of Culturing Stem Cells Using Double-Layer Composite Hydrogel Microcarrier

First of all, suspension solution of adipose stem cells containing 1×10⁷ cells/mL adipose stem cells (that is, the adipose stem cells were suspended in the culture fluid for adipose stem cells) and the double-layer composite hydrogel microcarrier were mixed with a volume ratio of 1:1 to form a stem cell hydrogel mixture, and then stood for 30 min at 37° C. to increase gelation extent.

Furthermore, after dropping 400 μL of the culture fluid for adipose stem cells on the surface of the stem cell hydrogel mixture, the stem cell hydrogel mixture was placed in an environment containing 5% carbon dioxide at 37° C. 200 μL of the culture fluid for adipose stem cells (suspension) was taken out and replaced with the fresh culture fluid for adipose stem cells every 2 days. The adipose stem cells were cultured until the double-layer composite hydrogel microcarrier cracked, and then, the adipose stem cells were collected using PBS, and other culture ingredients were removed.

It could be understood that, through the setting of the outer-layer hydrogel structure, physical stability could be provided to the inner-layer hydrogel structure, preventing the inner-layer hydrogel structure from swelling and cracking due to water absorption or structural collapse in the alkaline environment, and at the same time, avoiding excessive release and leakage of nutrient ingredients, maintaining the stable release of nutrient ingredients.

### Example 3—Functional Tests

First of all, the double-layer composite hydrogel microcarrier was manufactured according to abovementioned Example 1, and specific ingredients, weight percentages and the formation time (that is, mixing time of “2. Outer-Layer Hydrogel Structure and Double-layer composite hydrogel microcarrier”) of the outer-layer hydrogel structure were adjusted, the differences of the structural characteristics and content release efficiency between different double-layer composite hydrogel microcarriers were compared. Examples of each set of conditions were as follows:

TABLE 1
Conditions of Testing Groups

| | | | | Crosslinking |
|---|---|---|---|---|
| | | | | Time of |
| | Inner Layer (wt %) | | Outer | Outer Layer |
| Group | SA | CMC | Layer | (min) |
| SA-CMC@PDMA-1 | 1.5 | 1 | PDMA | 1 |
| SA-CMC@PDMA-2 | 0.5 | 2 | PDMA | 1 |
| SA-CMC@PDMA-3 | 0.5 | 2 | PDMA | 3 |
| SA-CMC@PAA-1 | 1.5 | 1 | PAA | 1 |

1. Gel morphology in Electron Microscopy

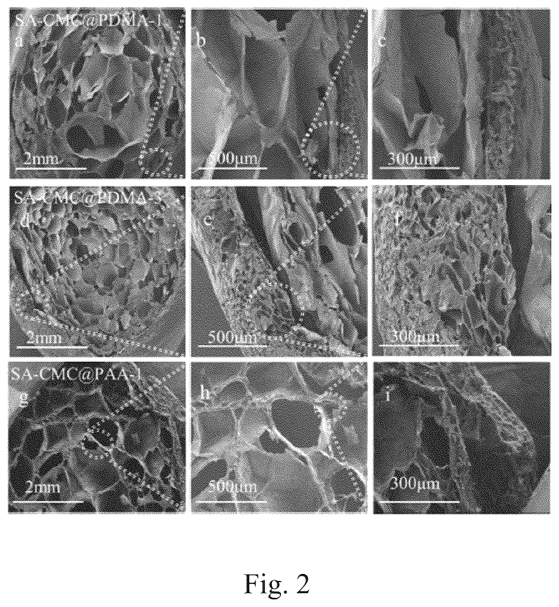

First, through a scanning electron microscope, the double-layer composite hydrogel microcarrier of each group was observed at different magnifications. Please refer to FIG. 2 for the results, in which a to c of the uppest row were SA-CMC@PDMA-1 group, d to f of the second row were SA-CMC@PDMA-3 group, and g to i of the lowest row were SA-CMC@PAA-1 group.

It was represented in FIG. 2 that the inner layer and the outer layer of the double-layer composite hydrogel microcarrier were respectfully represented as sheet structures connected to each other, the sheet structures were spaced apart from each other by three-dimensional holes, and a clear boundary between the inner layer and the outer layer was represented. Furthermore, different hole sizes between the inner-layer hydrogel structure and the outer-layer hydrogel structure were represented, and the hole diameter of the outer-layer hydrogel structure was smaller than the hole diameter of the inner-layer hydrogel structure.

In addition, by comparing FIG. c (SA-CMC@PDMA-1 group) and FIG. f (SA-CMC@PDMA-3 group), it could be found that as the crosslinking time of the outer layer increased from 1 min to 3 min, the thickness of the outer-layer hydrogel structure increased from about 100 μm to about 450 μm.

In addition, by comparing FIG. b (SA-CMC@PDMA-1 group) and FIG. e (SA-CMC@PDMA-3 group), it could be found that as the concentration of SA decreased, the hole diameters of the holes in the inner layer and the outer layer were significantly changed, in which along with the higher ratio of SA, the hole diameters of the inner-layer holes was greater.

According to FIG. 2, it could be understood that, the double-layer composite hydrogel microcarrier had a double-layer skeleton structure. The inner-layer hydrogel structure had a larger hole diameter, which was beneficial to adsorbing nutrient ingredients. The outer-layer hydrogel structure was relatively dense, which could provide physical support to the inner-layer hydrogel structure, avoid the collapse of the inner-layer hydrogel structure, and avoid excessive release of nutrient ingredients. Therefore, the double-layer composite hydrogel microcarrier could provide a scaffold for the three-dimensional growth of stem cells and retain nutrient ingredients for a long time, increasing the cultured amount of stem cells and prolonging the sustainable culture time. The relevant functions would be further verified below.

### 2. Water Swellability

For detecting water swellability of inner-layer hydrogel structure (single-layer hydrogel) only and double-layer composite hydrogel microcarrier, the inner-layer hydrogel structures (four groups, the concentration of SA and the concentration of CMC in each group were respectfully 0.5% SA/2.0% CMC, 1.0% SA/1.5% CMC, 1.5% SA/1.0% CMC, 2.0% SA/0.5% CMC) and double-layer composite hydrogel microcarrier (SA-CMC@PDMA-1 group, SA-CMC@PDMA-2 group, SA-CMC@PDMA-3 group, SA-CMC@PAA-1 group) were air dried naturally. An appropriate amount of the air-dried inner-layer hydrogel structure and the air-dried double-layer composite hydrogel microcarrier were weighed (hereinafter referred to as the sample), and a swelling test was performed. The process was as follows.

First, the sample was placed in an acidic solution with a pH of 1.2 (0.5M hydrochloric acid) for 2 hours, and then, converted to an alkaline solution with a pH of 7.4 (1M trishydroxymethylaminomethane) until the weight reached equilibrium or decreased (indicating the beginning of dissolution). During the process, the sample was taken out from the solution every 30 minutes, and the residual solution was removed and then weighed. The following formula was used to calculate the swelling ratio SR (swelling ratio) at each time point.

SR=[(m0−mt)/m0]

SR was the swelling rate of the double-layer composite hydrogel microcarrier, mt was the mass (swollen state) of the double-layer composite hydrogel microcarrier at time point t, and m0 was the initial mass of the hydrogel.

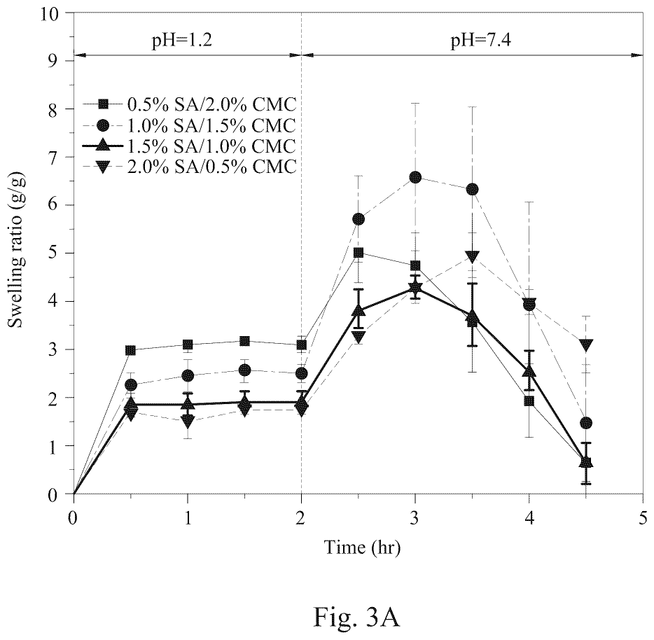

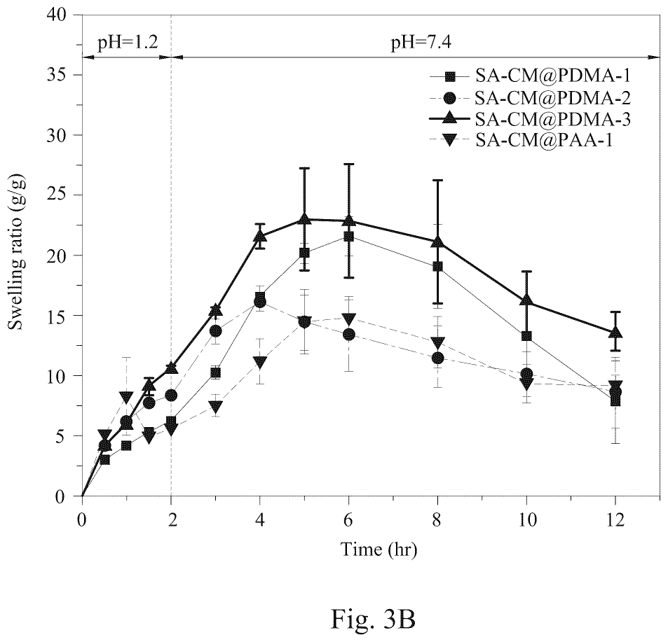

Please refer to FIG. 3A for the trend of the swelling rate of the inner-layer hydrogel structure obtained from the aforementioned test, and please refer to FIG. 3B for the trend of the swelling rate of the double-layer composite hydrogel microcarrier.

FIG. 3A represented water absorption swelling characteristics of different inner-layer hydrogel structures. The swelling ratio of the four inner-layer hydrogel structures in the acidic environment were lower because there were more carboxyl groups (—COOH) in SA and CMC, and in the acidic solution, the carboxyl groups were less free, the electrostatic repulsion between polymers was lower, the gel network structure was dense, and the hydrophilicity of SA and CMC decreased. Therefore, compared with the alkaline solution, the swelling ratio of the inner-layer hydrogel structure (single-layer hydrogel) was lower in the acidic solution. In addition, it was also observed that in acidic solution, as the decrease of the CMC concentration, the swelling ratio also decreases.

Relatively, in the alkaline environment, the swelling ratio increased with time, gradually increased and then decreased. Specifically, the carboxyl groups were in a free state (COO—), and electrostatic repulsion was formed between the carboxyl groups and increased with time, causing the single-layer hydrogel to swell and increase the network gap, changing the structure, increasing the hydrophilicity, absorbing water molecules, thereby increasing the swelling ratio. In the later stage of the reaction, as the structure changed, the ionic crosslinking formed by carboxyl groups and calcium ions gradually became unstable, and the concentration of carboxyl groups decreased, which lead to a weaker cross-linking reaction between carboxyl groups and calcium ions, causing the single-layer hydrogel to gradually collapse.

FIG. 3B represented water absorption swelling characteristics of double-layer composite hydrogel microcarriers with different hydrogel structures. Compared with the water absorption swelling time (the time from absorbing water to collapse) of the single-layer hydrogel (only inner-layer hydrogel structure) in FIG. 3A, which was 4.5 hours. FIG. 3B represented that the water absorption swelling time of the double-layer composite hydrogel microcarrier was significantly prolonged to 12 hrs, and the overall swelling ratio was higher than that of the single-layer hydrogel (that is, the inner-layer hydrogel structure of the double-layer composite hydrogel microcarrier). Although the inner-layer hydrogel structure was sensitive to pH, the outer-layer hydrogel structure was less sensitive to pH. Therefore, even in the alkaline environment, the outer-layer hydrogel structure could maintain the integrity of the gel structure and was not easy to collapse.

In addition, by comparing the SA-CMC@PDMA-2 group (outer layer cross-linking time was 2 hrs) and the SA-CMC@PDMA-3 group (outer layer cross-linking time was 3 hrs), it could be found that the collapse of the hydrogel was delayed by increasing the cross-linking time of the outer layer to increase the thickness of the outer-layer hydrogel structure.

By comparing SA-CMC@PDMA-1 group (1.5% SA/1.0% CMC) and SA-CMC@PDMA-2 group (0.5% SA/2.0% CMC), it could be found that the groups with reduced SA concentration had a lower swelling ratio after 4 hrs. Therefore, in the alkaline environment, reduction of SA concentration could be used as a pathway to prolong the release time of nutrient ingredients.

Finally, by comparing SACMC@PDMA-1 group (PDMA) and SA-CMC@PAA-1 group (PAA), it could be found that compared with PAA, PDMA used as the outer-layer hydrogel structure represented a higher swelling ratio and better water-absorbing swelling capacity.

### 3. Content Release Behavior

### 3.1. Testing Method

For testing the content release behavior of the double-layer composite hydrogel microcarrier of each group, the content release behavior was performed according to the following procedures.

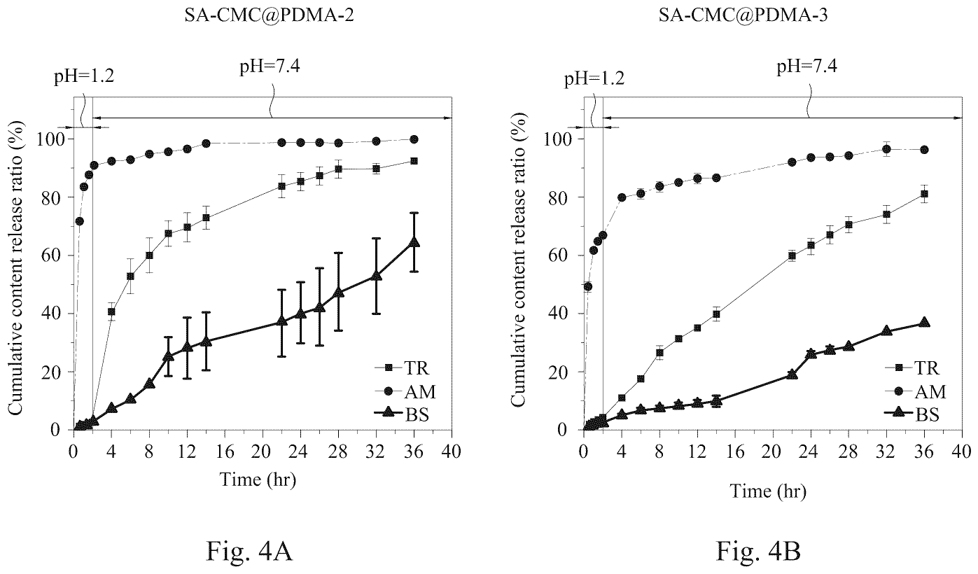

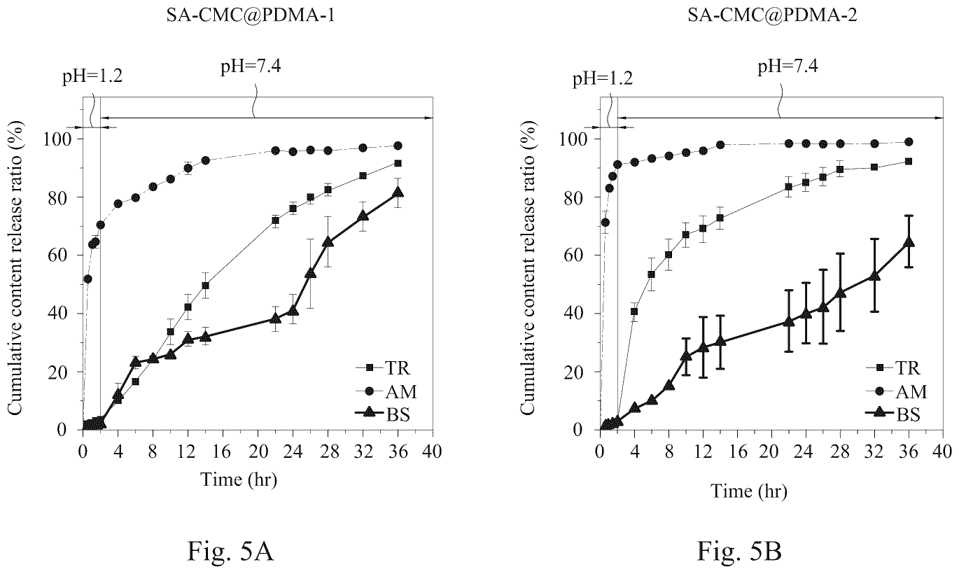

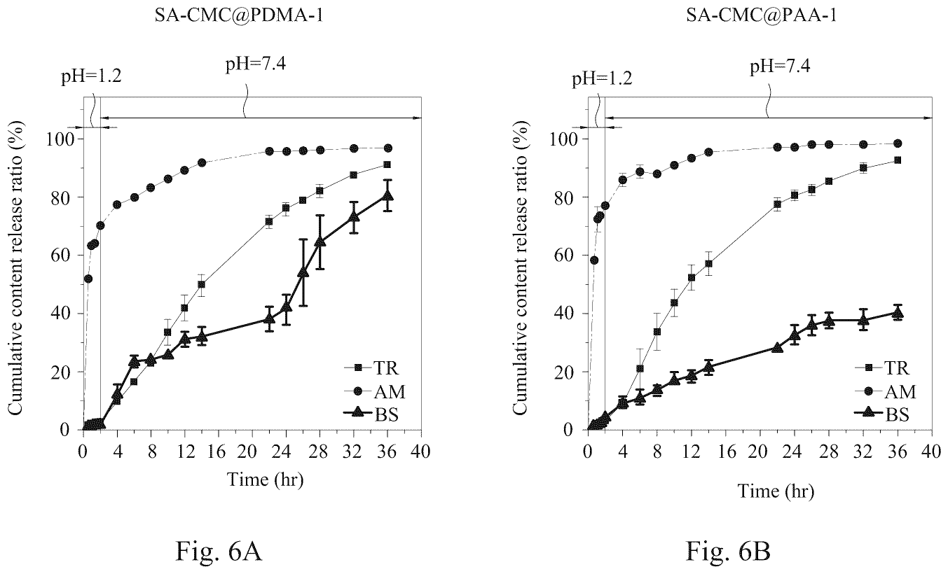

First of all, three contents with different molecular weights and solubility characteristics were selected to mix with the double-layer composite hydrogel microcarriers in different groups to make the double-layer composite hydrogel microcarrier carry each of the contents, in which tretinoin (TR) representing lipd soluble ingredient, ampicillin (AM) representing water soluble ingredient, and bovine serum protein (BS) representing ingredients in the culture fluid for stem cells were respectfully selected as contents, in which the concentration of tretinoin in the double-layer composite hydrogel microcarrier was 0.5 mg/L, the concentration of ampicillin in the double-layer composite hydrogel microcarrier was 100 μg/mL, and the weight percentage of bovine serum albumin in the double-layer composite hydrogel microcarrier was 5%. The double-layer composite hydrogel microcarrier containing the content was immersed into the acidic solution with pH of 1.2 for 2 hrs, and transferred to the weakly alkaline solution with pH of 7.4 at 36.5° C.±0.5° C. Furthermore, after weighing the double-layer composite hydrogel microcarrier, the double-layer composite hydrogel microcarrier was immersed into 100 mL of PBS, rotated and oscillated at an appropriate speed at a temperature of 36.5° C.±0.5° C. At a period time, a quantitative amount of PBS was extracted and the content concentration was measured using a UV spectrophotometer (TR was measured at a wavelength of 350 nm, AM was measured at a wavelength of 463 nm, and BS was measured at a wavelength of 595 nm), and an equal amount of PBS was added. Furthermore, according to the following formula, the cumulative content release ratio Qn of the double-layer composite hydrogel microcarrier was measured, and the results were organized in FIG. 4A to FIG. 6B.

$$QN ( \% ) = ( V 0 Cn + V C ( n - 1 ) ) / ( W ) \times 100$$

Cn and C(n−1) were the content concentrations of n (times) and n−1 (times) of sampling, V0 was the initial volume of PBS, V was the sampling volume, and W was the weight of the content loaded in the double-layer composite hydrogel microcarrier.

### 3.2. Result Analysis

In order to facilitate the understanding of influence of ingredients of the double-layer composite hydrogel microcarrier, weight percentage and outer layer cross-linking time to content release behavior, the follow-up would be discussed by comparing FIG. 4A (SA-CMC@PDMA-2) and FIG. 4B (SA-CMC@PDMA-3), FIG. 5A (SA-CMC@PDMA-1) and FIG. 5B (SA-CMC@PDMA-2), and FIG. 6A (SA-CMC@PDMA-1) and FIG. 6B (SA-CMC@PAA-1).

### 3.2.1. Comparison of Thickness of Outer Layers and Content Release Behavior

First of all, please refer to FIG. 4A and FIG. 4B. The difference between the two groups of double-layer composite hydrogel microcarriers was that the outer layer cross-linking time was prolonged from 2 min (SA-CMC@PDMA-2 group) to 3 min (SA-CMC@PDMA-3 group), making the thickness of the outer-layer hydrogel structure increase.

FIG. 4A and FIG. 4B represented that in the initial acidic environment, both double-layer composite hydrogel microcarriers had good sustained-release effects for tretinoin (TR) and bovine serum albumin (BS), which were lipid soluble, but didn't provide the same control effect for ampicillin (AM), which was water soluble. Since AM had a small molecular weight and was easily diffused into the solution, AM produced a severe release effect. In SA-CMC@PDMA-2 group, the release ratio in two hrs was up to about 90%, and in SA-CMC@PDMA-3 group with thicker outer layer, the release ratio in two hrs was about 65% since the diffusion resistance was increased, representing the thickness of the outer layer provided the effect of delaying release of water soluble ingredient, AM, in the acid environment.

In the weakly alkaline environment, the inner-layer hydrogel structure began to absorb water and swell, and the network structure collapsed, thereby gradually releasing tretinoin (TR) and bovine serum albumin (BS). In SA-CMC@PDMA-2 group, TR release ratio was more than 90% at 36 hrs, but for the release ratio of BS with a larger molecular structure, the release ratio only reached about 64%, representing the design of the double-layer structure provided the better sustained release effect of the ingredients in culture fluid for the stem cells (demonstrated by BS) than that of lipd soluble ingredient (demonstrated by TR). Relatively, for SA-CMC@PDMA-3 group with a thicker outer layer, the TM release ratio dropped to about 80% at 36 hrs, and the BS release ratio only reached 35%, indicating that in the weakly alkaline environment, the lipid soluble ingredient (TR) and the ingredients in the stem cell culture fluid (BS) were more effective in delaying and controlling release.

Therefore, according to FIG. 4A and FIG. 4B, it could be understood that by increasing the thickness of the outer layer, the diffusion resistance could be increased, thereby significantly delaying the release of the contents.

### 3.2.2. Comparison of Concentration of Inner Layer Composition and Content Release Behavior

Please refer to FIG. 5A and FIG. 5B. The difference of the double-layer composite hydrogel microcarriers between the two groups was that the weight percentages of the inner-layer polymers were different (SA-CMC@PDMA-1 group was 1.5% SA/1.0% CMC; SA-CMC@PDMA-2 group was 0.5% SA/2.0% CMC).

The results of FIG. 5A and FIG. 5B represented that in the initial acidic environment, the composition of the inner-layer hydrogel structure had no significant change in the sustained release effect of the double-layer hydrogel, and the lipid-soluble tretinoin (TR) and bovine serum albumin (BS) both have good sustained-release effects, while water-soluble amp (AM) still exhibits severe release effects, with a 2-hour rate as high as 70% to 90%. The results of FIG. 5A and FIG. 5B represented that, in the initial acidic environment, the composition of the inner-layer hydrogel structure had no significant change in the sustained release effect of the double-layer hydrogel, lipid soluble tretinoin (TR) and bovine serum albumin (BS) both had good sustained-release effects while water soluble ampicillin (AM) still exhibited severe release effects, with a 2-hour ratio as high as 70% to 90%.

In the weakly alkaline environment, there was no significant difference in the release ratio of TR and AM between the two groups. However, SA-CMC@PDMA-2 with a lower SA concentration (the hole diameter of SA-CMC@PDMA-2 illustrated in “1. Gel morphology in electron microscopy” was obviously smaller than that of SA-CMC@PDMA-1) had better control effect for BS release.

Compared with macromolecule BS, lipid soluble TR and water soluble ampicillin AM both belonged to small molecules. According to FIG. 5A and FIG. 5B, it could be found that the composition change of the inner-layer hydrogel structure had little impact on the small molecule content. However, reduction of the SA concentration delayed the diffusion rate of the macromolecule content and reduced the release ratio.

### 3.2.3. Comparison of Ingredients of Outer Layer Composition and Content Release Behavior

Please refer to FIG. 6A and FIG. 6B, the difference between the double-layer composite hydrogel microcarriers of the two groups was that the ingredient of the outer layer polymer was different (SA-CMC@PDMA-1 group was PDMA; SA-CMC@PAA-1 group was PAA). The difference between the two groups of the double-layer composite hydrogel microcarriers was that the ingredient of the outer-layer polymer was different (SA-CMC@PDMA-1 group was PDMA; SA-CMC@PAA-1 group was PAA).

The results of FIG. 6A and FIG. 6B represented that there was no significant difference in the release ratio of lipid soluble tretinoin (TR) and water soluble ampicillin (AM) in the two groups of double-layer composite hydrogel microcarriers. However, the release rate of bovine serum albumin (BS) was significantly reduced when the ingredient of the outer-layer polymer, PDMA, was replaced with PAA that had lower swelling ratio and providing smaller hole diameter.

That is, compared with selection of PDMA, when PAA was selected as the ingredient of the outer-layer hydrogel structure, the diffusion rate of the macromolecule content was prolonged and the release ratio was reduced although little impact was provided on the small molecule content.

### 4. Strength of Structure

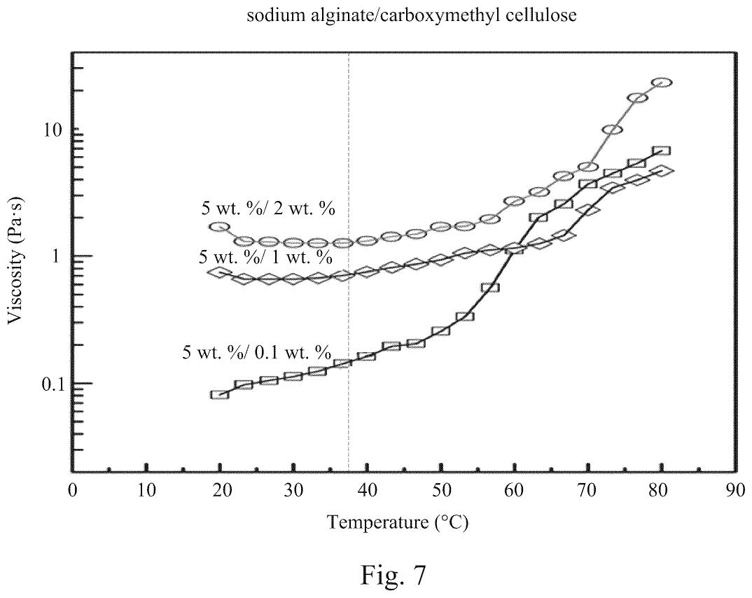

In order to test the relationship between the content of specific ingredient in the gel structure and the structural strength, the single-layer gel structure was prepared basically according to the steps similar to point 1.1 of Example 1 and according to the formula in Table 2 below, and the relationship between viscosity and temperature of the single-layer gel structure was tested. The results were represented in FIG. 7.

TABLE 2
Conditions of Tested Groups

| | Sodium Alginate (SA) | Carboxymethyl Cellulose |
|---|---|---|
| Group | (wt. %) | (wt. %) |
| 1 | 5 | 2 |
| 2 | 5 | 1 |
| 3 | 5 | 0.1 |

FIG. 7 represented that at 37.5° C., as the concentration of carboxymethyl cellulose (CMC) increased, the viscosity of the gel structure increased. It could be understood that as the viscosity of the gel structure increased, the structural strength increased. Therefore, as the CMC concentration increased, the structural strength of the gel structure also increased.

It should be emphasized that when the viscosity approached or was higher than 1 Pa·s (for example, SA of group 1 was 5 wt % and CMC of group 1 was 2 wt %, and SA of group 2 was 5 wt % and CMC was 1 wt %), the structural strength of the gel structure could be suitable for culturing stem cells and was not easy to crack.

Relatively, when the viscosity of the gel structure was too low (for example, SA of group 3 was 5 wt % and CMC of group 3 was 0.1 wt %), the risk of cracking of the gel structure was higher when the gel structure was used to culture stem cells.

Therefore, when the gel structures of groups 1 and 2 were used as the inner-layer hydrogel structures (the weight percentage of CMC was greater than 1 wt %), better structural strength could be provided to the double-layer composite hydrogel microcarrier for use as growth scaffold of stem cells.

### 5. Culture Efficiency of Stem Cells

First, SA-CMC@PDMA-1 (SA was 1.5% and CMC was 1%) and SA-CMC@PDMA-2 (SA was 0.5% and CMC was 2%) in Table 1, two groups had double-layer composite hydrogel microcarriers with different SA and CMC weight ratios, were selected, and the adipose stem cells were cultured according to the method similar to Example 2. At the same time, the culture conditions were divided into groups with or without the addition of the nutrient ingredient, bovine serum albumin (called BS or BSA), and the adipose stem cells were observed simultaneously.

Through the following two parts of analysis, it was observed whether the double-layer composite hydrogel microcarriers with different ingredient ratios affected the growth efficiency of the adipose stem cells when the double-layer composite hydrogel microcarriers were used to culture the adipose stem cells.

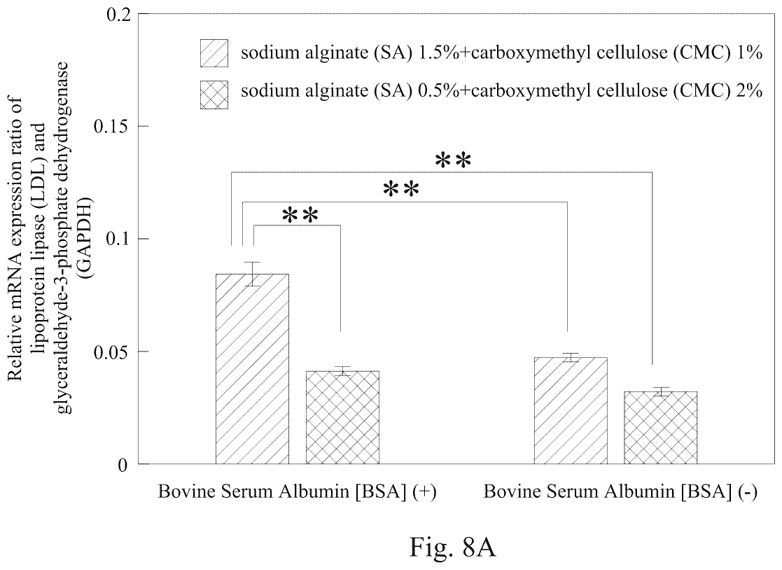

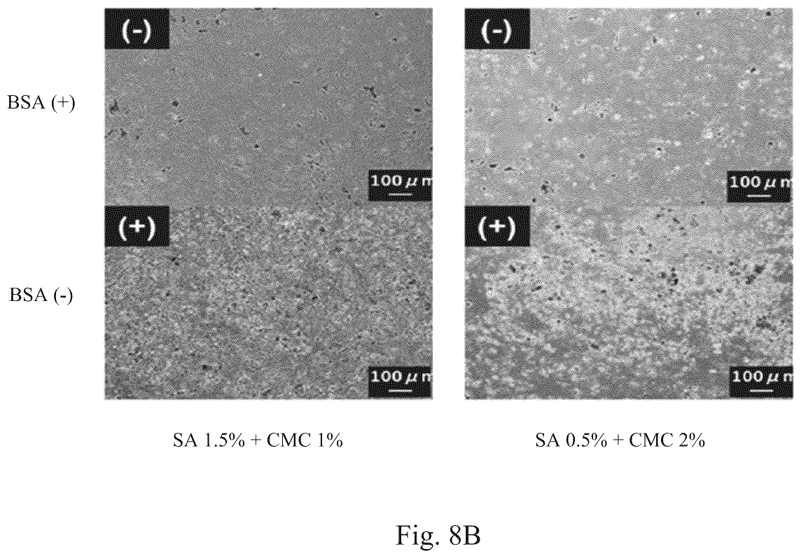

The first part was mRNA analysis. When the adipose stem cells were cultured for 7 days, mRNA expression ratio of the unique protein of adipose stem cells (lipoprotein lipase, LPL) and the common protein of cells (glyceraldehyde-3-phosphate dehydrogenase, GAPDH) in the adipose stem cells was analyzed to determine the growth of the adipose stem cells. The larger the ratio was, the more the adipose stem cells were. The results were shown in FIG. 8A.

FIG. 8A represented that, regardless of whether BSA was added, compared with SA-CMC@PDMA-2 (SA was 0.5% and CMC was 2 wt %), groups of SA-CMC@PDMA-1 (SA1.5%+CMC1%) all had better growth efficiency of the adipose stem cells.

The second part was the observation under the microscope field of view. The number of growth cells of each group of the adipose stem cells was observed under the microscope field of view when they were cultured for 7 days, in which the adipose stem cells were recognized by CD44 monoclonal antibody, and stained with Oil Red O to appear purple-red. Therefore, the higher the purple-red ratio was, the higher the amount of adipose stem cells was. Please refer to FIG. 8B for the results.

FIG. 8B represented that when adding macromolecular nutritional molecules (BSA), compared with SA-CMC@PDMA-2 (SA was 0.5% and CMC was 2 wt %), the ratio of the adipose stem cells in SA-CMC@PDMA-1 (SA 1.5%+CMC 1%) was higher. That is, when adding nutrient ingredients, the weight ratio condition of SA-CMC@PDMA-1 could be used to achieve better culture efficiency of the stem cells.

A method of culturing a stem cell by using a double-layer composite hydrogel microcarrier is provided in some embodiments of the present disclosure. Through the design of inner-layer hydrogel structure and the outer-layer hydrogel structure, it can provide physical support to the inner-layer hydrogel structure, the structure can be prevented from being affected by pH, diffusion resistance is increased, and the release of nutrient ingredient is delayed. By defining the weight ratio of sodium alginate and carboxymethyl cellulose and the position of the nutrient ingredient, the hole diameter of the inner-layer hydrogel structure can be controlled, the release ratio of the nutrient ingredient from the double-layer composite hydrogel microcarrier is regulated, allowing the stem cells to stably absorb the nutrient ingredient, thereby increasing the growth rate of the stem cells. By controlling the weight percentage of carboxymethyl cellulose, the structural strength of the double-layer composite hydrogel microcarrier can be ensured, so as to make the double-layer composite hydrogel microcarrier serve as growth scaffold for the stem cells.

Although the present invention has been disclosed in the above embodiments, it is not intended to limit the present invention. Anyone familiar with this technique can make various changes and modifications without departing from the spirit and scope of the present invention. The scope of protection of the invention shall be subjected to the scope of appended claims.

What is claimed is:

1. A method of culturing a stem cell by using a double-layer composite hydrogel microcarrier, comprising:
providing a stem cell, a double-layer composite hydrogel microcarrier and a nutrient ingredient,
wherein the double-layer composite hydrogel microcarrier comprises:
an inner-layer hydrogel structure, formed by ionic crosslinking of an inner-layer polymer through an inner-layer cross-linker, wherein the inner-layer polymer comprises a first inner-layer polymer and a second inner-layer polymer, the first inner-layer polymer is sodium alginate, the second inner-layer polymer is carboxymethyl cellulose, a weight ratio of the sodium alginate and the carboxymethyl cellulose is 3:2, and a weight percentage of the carboxymethyl cellulose is greater than 1% based on 100% by weight percentage of the inner-layer hydrogel structure; and
an outer-layer hydrogel structure, formed by covalent crosslinking of an outer-layer monomer through an outer-layer cross-linker, wherein the outer-layer hydrogel structure encapsulates the inner-layer hydrogel structure,
wherein the nutrient ingredient is located inside the inner-layer hydrogel structure;
mixing the stem cell, the double-layer composite hydrogel microcarrier and the nutrient ingredient to obtain a gelation culture medium; and
adding a culture fluid to the gelation culture medium.

2. The method of claim 1, wherein the stem cell comprises an embryonic stem cell, a hematopoietic stem cell, a mammary stem cell, a mesenchymal stem cell, an endothelial stem cell, a neural stem cell, an olfactory stem cell, an adipose stem cell or a combination thereof.

3. The method of claim 1, wherein
the inner-layer hydrogel structure is represented as a plurality of inner sheet structures, and the plurality of inner sheet structures are connected to each other and separated from each other by a plurality of inner-layer holes; and
the outer-layer hydrogel structure is represented as a plurality of outer sheet structures, and the plurality of outer sheet structures are connected to each other and separated from each other by a plurality of outer-layer holes, wherein a hole diameter of each of the plurality of outer-layer holes is less than a hole diameter of each of the plurality of inner-layer holes.

4. The method of claim 1, wherein the inner-layer hydrogel structure is represented as an interpenetrating network of the first inner-layer polymer and the second inner-layer polymer.

5. The method of claim 1, wherein the inner-layer polymer and the inner-layer cross-linker have opposite electrical properties.

6. The method of claim 1, wherein the outer-layer monomer comprises N,N-dimethylacrylamide, acrylamide or a combination thereof.

7. The method of claim 1, wherein the outer-layer cross-linker comprises N,N′-methylenebisacrylamide.

8. The method of claim 1, wherein a weight percentage of the sodium alginate is from 0.1% to 5% based on 100% by weight percentage of the inner-layer hydrogel structure.

9. The method of claim 1, wherein a weight percentage of the carboxymethyl cellulose is from 1% to 5% based on 100% by weight percentage of the inner-layer hydrogel structure.

10. The method of claim 1, wherein the nutrient ingredient comprises growth factor, tretinoin, ampicillin, bovine serum albumin or a combination thereof.

11. A method of for culturing a stem cell by using a double-layer composite hydrogel microcarrier, comprising:
providing a stem cell, a double-layer composite hydrogel microcarrier and a nutrient ingredient, wherein the nutrient ingredient comprises macromolecular protein with a molecular weight of at least 500 g/mol,
the double-layer composite hydrogel microcarrier comprises:
an inner-layer hydrogel structure, formed by ionic crosslinking of an inner-layer polymer through an inner-layer cross-linker, wherein the inner-layer polymer comprises a first inner-layer polymer and a second inner-layer polymer, the first inner-layer polymer is sodium alginate, the second inner-layer polymer is carboxymethyl cellulose, a weight ratio of the sodium alginate and the carboxymethyl cellulose is 3:2; and
an outer-layer hydrogel structure, formed by covalent crosslinking of an outer-layer monomer through an outer-layer cross-linker, wherein the outer-layer hydrogel structure encapsulates the inner-layer hydrogel structure,
wherein the nutrient ingredient is located inside the inner-layer hydrogel structure;
mixing the stem cell, the double-layer composite hydrogel microcarrier and the nutrient ingredient to obtain a gelation culture medium; and
adding a culture fluid to the gelation culture medium.

12. The method of claim 11, wherein the stem cell comprises an embryonic stem cell, a hematopoietic stem cell, a mammary stem cell, a mesenchymal stem cell, an endothelial stem cell, a neural stem cell, an olfactory stem cell, an adipose stem cell or a combination thereof.

13. The method of claim 11, wherein
the inner-layer hydrogel structure is represented as a plurality of inner sheet structures, and the plurality of inner sheet structures are connected to each other and separated from each other by a plurality of inner-layer holes; and
the outer-layer hydrogel structure is represented as a plurality of outer sheet structures, and the plurality of outer sheet structures are connected to each other and separated from each other by a plurality of outer-layer holes, wherein a hole diameter of each of the plurality of outer-layer holes is less than a hole diameter of each of the plurality of inner-layer holes.

14. The method of claim 11, wherein the inner-layer hydrogel structure is represented as an interpenetrating network of the first inner-layer polymer and the second inner-layer polymer.

15. The method of claim 11, wherein the inner-layer polymer and the inner-layer cross-linker have opposite electrical properties.

16. The method of claim 11, wherein the outer-layer monomer comprises N,N-dimethylacrylamide, acrylamide or a combination thereof.

17. The method of claim 11, wherein the outer-layer cross-linker comprises N,N′-methylenebisacrylamide.

18. The method of claim 11, wherein a weight percentage of the sodium alginate is from 0.1% to 5% based on 100% by weight percentage of the inner-layer hydrogel structure.

19. The method of claim 11, wherein a weight percentage of the carboxymethyl cellulose is greater than 1% based on 100% by weight percentage of the inner-layer hydrogel structure.

20. The method of claim 19, wherein the weight percentage of the carboxymethyl cellulose is from 1% to 5% based on 100% by weight percentage of the inner-layer hydrogel structure.
